(19) United States
(12) Patent Application Publication
(10) Pub. No.: US 2002/0162134 A1
(43) Pub. Date: Oct. 31, 2002

(54) Primordial germ cell-based germ line production of birds

(76) Inventors: Alexander Baguisi, Grafton, MA (US); Karl M. Ebert, Millbury, MA (US)

Correspondence Address: MINTZ, LEVIN, COHEN, FERRIS,, GLOVSKY and POPEO, P.C., One Financial Center, Boston, MA, 02111, US

(21) Appl. No.: 10/044,006

(22) Filed: Jan. 11, 2002

Related U.S. Application Data
(60) Provisional application No. 60/269,442, filed on Feb. 16, 2001.

(51) Int. Cl. A01K 67/027

(52) U.S. Cl. 800/19; 800/21

(57) ABSTRACT

The invention features an isolated avian gonadal cell, e.g., an ovarian cell or a testes cell, containing a heterologous nucleic acid as well as a method of introducing a nucleic acid molecule into the genome of an avian species, by contacting a population of isolated gonadal cells derived from a chick embryo with the nucleic acid molecule to yield transfected gonadal cells, and transferring the transfected gonadal cells to a fertilized avian egg

This application claims priority to U.S. provisional patent application No. 60/269,442, filed on Feb. 16, 2001, the entire contents of which is hereby incorporated by reference.

### BACKGROUND OF THE INVENTION

The invention relates to methods of genetically manipulating an avian genome.

Extensive effort has been directed at developing better and more efficient methods for producing transgenic animals for commercial and research purposes. Recent developments in the field described the use of intracytoplasmic sperm injection (ICSI) for sperm-mediated transgenesis (Perry et al. 1999, Science 284: 1180-1183). Some researchers have reported the production of cloned animals by nuclear transfer in mammals, e.g., sheep (Wilmut etal. 1997, Nature 385:810-813), cows (Cibelli et al, 1998, Science 280:1256-1258), mice (Wakayama et al., 1998 Nature 394: 369-394) and goats (Baguisi et al. 1999, Nature Biotechnology 17:456-461) using somatic cells as donor karyoplast. However, these techniques are still in their early developmental stage and may be difficult to adapt to the uniquely different reproductive system of birds.

### SUMMARY OF THE INVENTION

The invention features an isolated avian gonadal cell, e.g., an ovarian cell or a testes cell, containing a heterologous nucleic acid. The cell is preferably an embryonic cell. The term “avian” refers to any avian species, including but not limited to, chicken, turkey, duck, goose, quail, and pheasant.

The invention also includes a method of introducing a nucleic acid molecule into the genome of an avian species, by contacting a population of isolated gonadal cells derived from a chick embryo with the nucleic acid molecule to yield transfected gonadal cells, and transferring the transfected gonadal cells to a fertilized avian egg. The nucleic acid molecule is heterologous, i.e., it is derived from a breed or species which differs from the breed or species from which the gonadal cell is derived. Transfected gonadal cells are used then used to produce transgenic birds.

The population of isolated gonadal cells contains at least 0.5% primordial germ cells (PGCs), more preferably at least 1% primordial germ cells, and even more preferably at least 50% primordial germ cells. Gonadal PGCs are isolated, i.e., separated, from other cells, e.g., stromal gonadal cells, with which they naturally-occur in a tissue. Preferably, the population is at least 80%, more preferably, 90%, more preferably 95%, and most preferably 99-100% gonadal PGCs. The PGCs are transfected with heterologous DNA either before or after purification from other non-PGC cells. Gonadal avian PGCs are cells that exist in an embryonic chick which can give rise to oocytes and sperm cells.

The chick embryo from which the gonadal PGCs are obtained is preferably at an embryonic stage of greater than 27 of the developing chick embryo (Hamburger & Hamilton, 1951 J Morphol 88: 49-92). For example, the chick embryo is at an embryonic stage of 29-36 of development. Preferably, the chick embryo from which the gonadal cells are obtained has been incubated for at least 6.5 days, and more preferably 7.5 days (stage 29-36 of development) at the time of recovery. Gonadal PGCs obtained from chick embryos at least 6.5 d but less than 15 d of developmental age retain migratory capacity. For example, day 8, 9, 10, 11, 12, 13, or 14 gonadal PGCs are transferred to recipient eggs and retain migratory capability.

Transfected donor gonadal cells are derived from the same or different breed of bird compared to the breed from which the recipient egg is obtained. Similarly, the transfected donor gonadal PGCs are isolated from the same or different species of bird compared to the species from which the fertilized recipient avian egg is obtained. The fertilized recipient avian egg is between stage 7-8 of development, i.e., the recipient egg has been incubated for at least 12 hours. Alternatively, the fertilized avian egg is between stage 13-19 of development.

Eggs which are unincubated include those which have just been laid or those which have been stored at a temperature less than 37° C. For example, unincubated eggs include those which have been stored at a storage temperature of approximately 60° F., at room temperature, or in the cold (e.g., at 4° C.) from the time just after laying to the time of incubation. Incubation refers to the time at which an egg is exposed to temperature conducive to development (e.g., 37-38° C. or a temperature tailored to the requirements of the breed or species of bird). The stage of development is determined chronologically or by visual examination of the state, e.g., size and morphology) of embryonic tissues.

Preferred promoters to regulate expression of heterologous sequences include tissue-specific promoters, e.g., those which direct expression of the transgene in oviductal cells of the chicken. Transgene expression in oviductal cells leads to accumulation of the trangene product in the albumen of the egg. For example, the ovalbumin promoter known in the art is used to direct expression in oviductal cells. Alternatively, a promoter directs expression of the transgene in liver tissue. A vitellogenin promoter is used to direct expression of a heterologous polypeptide (encoded by the transgene) to the liver and secretion into the blood stream. For example, the transgene sequence operably linked to a vitellogenin promotoer encodes a light chain and heavy chain of an antibody, which confers disease resistance. Introduction of such a construct is useful for breed improvement. Promoters which direct expression in blood, muscle, feathers, or other tissues are also used. Tissue-specific enhancer may also be used to augment expression in a preferred target tissue. Alternatively, promoters which are not tissue-specific are used to direct expression systemically.

The transgene construct contains one or more promoters. For example, the construct contains two promoters, e.g., a first promoter operably linked to a sequence which encodes a transient marker gene (expression of which allows selection of transformed PGCs) and a second promoter which directs expression of the transgene in a tissue-specific manner.

Preferred transgenes include insulin and antibody molecules or fragments thereof. For example, the transgene encodes an intact heterodimeric monoclonal antibody, or an an immunologically-active antibody fragment, e. g., a Fab or (Fab)2 fragment, an engineered single chain Fv molecule, or a chimeric molecule, e.g., an antibody which contains the binding specificity of one antibody, e.g., of murine origin, and the remaining portions of another antibody, e.g., of human origin. Transgenes, e.g., antibody encoding genes, the gene for human or porcine insulin (or insulin from another mammal) is operably linked to an ovalbumin promoter to allow accumulation of insulin in the egg white fraction of an egg. For production of heterodimeric antibody molecules, a light chain encoding sequences are operably linked to an ovalbumin promoter, and heavy chain sequences are operably linked to an ovalbumin promoter for expression of both chains in oviductal cells. The transgene product is then purified from the egg using methods known in the art. Expression of antibody molecules is preferably targeted for accumulation in the egg white fraction of an egg (e.g., using an ovalbumin promotoer for expression in oviductal cells).

Donor PGCs are obtained from sex differentiated gonads and are therefore segregated by sex. Donor PGCs stocks are sex-matched with the recipient egg. Such a strategy insures favorable fertility rates and high germline expression of the donor PGCs (tranfected or untransfected). The sex of the recipient egg is hormonally controlled, e.g., by introducing testerone into the egg to generate a male chick or by introducing estrogen or follicle stimulating hormone into the egg to generate a female chick. Untransfected PGCs of one species are used to generate birds of another species.

Transgenic birds in which a gene has been disrupted (i.e., a “knockout” transgenic) are also generated using the methods described above. To produce a knockout bird, a knockout construct is made containing sequences complementary to sequences in the endogenous gene. The sequences in the transgene construct undergo homologous recombination with the target sequences resulting in disruption of the gene. Disruption of the gene leads to production of a non-functional gene product or results in little or no production of the gene product. Genes which are involved in pathological conditions are disrupted in this manner. The resulting knockout transgenic bird may be used as an animal model for the disease state. Such knockout transgenic birds are produced using an isolated avian gonadal cell, which contains a genetic disruption of an endogenous gene, e.g., a disruption which inhibits production of a functional gene product.

The invention includes an avian egg containing a xenogeneic PGC, e.g., a gonadal PGC. By xenogeneic is meant that the PGC and the avian (recipient) egg are of different species. For example, a chicken egg contains a PGC from an emu or an avian species other than a chicken. The invention also encompasses a chicken egg of one breed containing a gonadal PGC from another breed of chicken. An avian egg containing an isolated transgenic gonadal cell, e.g., a gonadal PGC transfected with a heterologous DNA, is also within the invention.

An isolated population of sex-determined PGCs, e.g., a population of male gonadal PGCs or a population of female gonadal PGCs, is also within the invention. Preferably, the population of male gonadal PGCs contains less than 20%, preferably less than 10%, preferably less than 5%, and more preferably less than 1% female gonadal PGCs. Similarly, the population of female gonadal PGCs contains less than 20%, preferably less than 10%, preferably less than 5%, and more preferably less than 1% male gonadal PGCs.

Other features and advantages of the invention will be apparent from the following detailed description, the drawings, and the claims.

### BRIEF DESCRIPTION OF THE DRAWINGS

FIG. 1 is a flow chart showing the procedure for isolating donor PGCs and preparing recipient eggs for transfer of donor PGCs.

FIGS. 2A and 2B are photographs of chicks generated from two pairs of breeding recipients that received donor PGCs.

FIGS. 3A and 3B are photographs of chicks generated from the transfer of both Rhode Island Red and Barred Rock PGCs to White Leghorn recipients. FIG. 3A shows pure donor derived chicks generated from Rhode Island Red males bred to Barred White Rock females (commercially sold as Redi-Link Cross). The females are black and males are barred. FIG. 3B shows pure donor derived chicks. Sex-linked chicks generated from the mating of Rhode Island Red males to Barred Rock females. The mating results in cockerel chicks with a white spot on the head and females do not (the popular commercial Red-Rock Cross). White chicks were derived from the endogenous germ cells of the White Leghorn recipients.

FIG. 4 is a photograph of a transgenic chick embryo showing expression of test transgene beta galactosidase. PGCs were transfected with the transgene and transferred to a recipient egg. The recipient chick embryo was recovered at day 10 of incubation to test for transgene expression. Beta galactosidase expression was detected in the mesonephros-gonadal region. Donor PGCs were transfected in vitro using lipids and transferred to the germinal crescent region of a 48 h incubated egg that was partially sterilized with 75 micrograms of busulphan 24 h prior to PGC transfer. Beta galactosidase expression was under the control of the Cytomegalovirus promoter.

### DETAILED DESCRIPTION OF THE INVENTION

Avian gonadal cells are genetically engineered and used to introduce heterologous nucleic acids into the avian genome. The methods are used to improve breed quality, produce avian models of non-avian diseases (e.g., human diseases), confer disease resistance to birds, and to produce recombinant proteins for pharmaceutical and other uses. The use of gonadal cells to mediate DNA transfer allows the production of birds across the different breeds and between species of birds for conservation of endangered species using the chicken as the “universal recipient”.

Isolated avian gonadal PGCs are used to produce transgenic animals in the avian species. Heterologous nucleic acids are introduced into the PGCs to yield stable transformed germ cells.

The methods described herein are applicable to all birds, and not limited only to chickens, turkeys, pheasants, ducks and geese. The overall efficiency of the procedure depends upon the timing of cell isolation, time and site of transfer, sex pre-selection of the donor PGCs, and the sterilization of the recipient embryo to maximize donor PGC population of the recipient gonads.

Standard genetic engineering technologies are used to produce transgenic animals. Several rounds of transfection of gonadal PGCs maybe carried out to increase gene transfer efficiency. Donor cells are purified before or after nucleic acid transfer. For example, PGCs are transfected in vitro and then purified prior to transfer into a recipient egg. The transfected (or transgenic gonadal PGCs are used to generate germline transgenic chickens for the production of pharmaceuticals in birds and eggs.

The sequence or structure of the transgene does not affect the success or efficiency of gene transfer. The promoter which regulates expression of the transgene is benign or tissue specific. Transgenic proteins or polypeptides are targeted for expression primarily in oviductal cells such that the gene product can be isolated from the egg after oviposition (laying of the egg). Use of tissue-specific promoters and/or enhancers allow expression in other tissues, including feathers, skin, and muscles.

Additionally, the method can also be used for the generation of other avian species using the chicken/egg as the “universal recipient” for production of other bird species in chickens and conservation of endangered bird species.

Uses of Genetically Modified Avian PGCs The cells and methods described herein are useful to produce germline birds from donor PGCs. The donor PGCs are transfected with heterologous nucleic acid and purified (or vice versa) so that an isolated population of transgenic PGCs are transferred to the recipient egg. Further enrichment of transfected PGCs is accomplished using a marker based cell sorter.

For agricultural applications, donor PGCs are transfected with a transgene that improves the quality of the bird or the meat and eggs the bird produces. For example, DNA encoding genes which confer disease resistance or a growth advantage are transferred to create animals with desirable market characteristics.

Therapeutic or pharmaceutical proteins or polypeptides are made by transfecting gonadal PGCs with DNA encoding the desired polypeptide, transferring the PGCs to a recipient egg, and incubating the egg under conditions suitable for the generation of germline chimera and subsequently transgenic birds for the production of the desired polypeptide. Therapeutic proteins are targeted for expression in the yolk or albumen of the egg or in the meat, feathers, skin, blood and other parts of the bird. The desired polypeptide is then isolated from the tissue or fluid (e.g., egg albumen) using methods known in the art.

The invention is also useful to produce a breed of bird which differs from the recipient breed (but is still within the same species, e.g., chicken). For example, the donor PGCs are derived from a Rhode Island Red breed and transferred to an egg of another breed of chicken (e.g., a Plymouth Rock). Common American breeds of chickens include Plymouth Rock, Dominiques, Wyandottes, Rhode Island Reds, Rhode Island Whites, Buckeyes, Chanteclers, Jersey Giants, Lamonas, New Hampshires, and Delawares.

Use of Isolated Avian Gonadal PGCs in Non-transgenic Applications

Isolated but untransfected gonadal PGCs are used to produce cross-species birds. For example, a quail is produced from an egg generated by a chicken. Thus, the methods of the invention provide for the use of a chicken or other domesticated bird as a “universal recipient” to produce cross-species birds for the conservation of endangered species. The advantage of such a production approach is that since the chicken is a non-seasonal highly productive domesticated bird, endangered birds are reliably produced without the seasonal or geographic limitations associated with endangered species. For example, the methods are used to produce rare or endangered bird such as the Houbarra Bustard, an endangered bird native to the Arabian peninsula. Other endangered birds include pheasants, quails, parrots and macaws, all of which are threatened by habitat loss and exploitation by hunters and traders. The methods are particularly useful to produce birds which are naturally seasonal breeders, e.g., pheasants or turkeys, or birds that do not breed in captivity. Albatross and petrels, which naturally breed on small oceanic islands, can also be reproduced by transfer of gonadal PGCs to a chicken recipient. Many species of rails, cranes and kagus are also at high risk because they are very slow-breeding animals, making them extremely vulnerable to disturbance of nesting grounds and wintering areas. Songbirds, which account for almost 60 percent of all bird species, have a slightly below-average risk of extinction, but some species, including those in American grasslands, are in serious decline. However, the methods are applicable to any other avian species, i.e., a non-chicken egg can also be used as a recipient to allow interspecies generation of birds. The developmental age at which sex differentiation of gonads occurs is determined by visual inspection for each species of bird.

### EXAMPLE 1

### Donor PGCs

Freshly laid (or unincubated) fertilized eggs are incubated for up to 10 days in a humidified egg incubator at 37-38° C. or at a temperature conducive to egg development. Egg incubation requirements for various species of birds is shown in Table 1.

TABLE 1
Egg Incubation Requirements for Avian species

| | Bobwhite | | | | | | | Chicken | |
|---|---|---|---|---|---|---|---|---|---|
| Requirements | Quail | Ostrich | Emu | Duck | Goose | Pheasant | Peafowl | Bantam | Turkey |
| Incubation | 23-24 | 42-48 | 48-50 | 28 | 28-34 | 23-28 | 28-30 | 21 | 28 |
| Period (days) | | | | | | | | | |
| Temperature | 99.5 | 96.5-97 | 96.5-97 | 99.5 | 99 | 99.5 | 99.5 | 99.5 | 99 |
| (F.) | | | | | | | | | |
| Humidity (wet | 84-86 | 70 | 70 | 84-86 | 86-88 | 86-88 | 83-85 | 85-87 | 83-85 |
| bulb, F) | | | | | | | | | |
| Final day of | 21 | 38-40 | 44-46 | 25 | 25 | 21 | 25 | 19 | 25 |
| egg rotation | | | | | | | | | |
| Temperature | 99 | 96-96.5 | 96-96.5 | 98.5 | 99 | 99 | 98.5 | 99 | 98.5 |
| during final 3 | | | | | | | | | |
| days of | | | | | | | | | |
| incubation (F.) | | | | | | | | | |
| Humidity | 90-94 | 75 | 75 | 90-94 | 90-94 | 92-95 | 90-94 | 90-94 | 90-94 |
| during final 3 | | | | | | | | | |
| days of | | | | | | | | | |
| incubation (F) | | | | | | | | | |

Gonads were harvested from the eggs between days 6-10 (stage 29-36, Hamburger & Hamilton, 1951 J Morphol 88: 49-92). Alternatively, the gonads are harvested between days 7-8 (stage 31-34). The data indicate that from day 7 of incubation, morphological differences between the sexes were identifiable allowing sex selection (females have a bigger left gonad while males have similarly sized pair of gonads). The avian embryo has received neurotrophic signals for sex differentiation and exhibit differences between ovarian and testis development. The PGCs retain migratory capacity and are remain responsive to chemoattractant factors which allow migration of the PGCs to the gonads of a developing embryo, e.g., in the recipient egg. The gonads were grouped by sex and dispersed by standard trypsinization procedure and cultured in tissue culture plates until they attach. Sex selection is carried out at the time of collection of the gonadal PGCs prior to transfer of the cells into a recipient egg.

The PGCs can be transfected together with the other gonadal stromal cells or isolated prior to transfection using a ficoll density gradient (Yasuda etal, 1992, J. Reprod. Fert. 96: 521-528). Further purification is accomplished by short-term culture (15-30 minutes) of the isolated cells. The gonadal stromal cells attach more rapidly to plastic, e.g., a tissue culture plate, than the PGCs, thus allowing purification of gonadal PGCs to approximately 90% or highter. The gonadal PGCs are transfected using Lipids (1-2 μg/ml DNA) for 3-4 hours or by electroporation at 250 volts and between 750-950 microfarads with 20-50 mg/ml of DNA. Alternatively, the PGCs are transfected using calcium phosphate and other methods known to the art for introducing nucleic acid sequences. Optionally, the cells are transfected more than once to increase the transfection rate. The PGCs are optionally cultured short term of up to 4 days in vitro without loss of migratory capability and without differentiation, allowing for several transfection repetitions and selection. Following short-term culture, the PGCs are separated from the stromal cells using standard methods. The transfected PGCs are further isolated by antibiotic selection, e.g., if the transgene construct carries a marker encoding antibiotic resistance. Other methods of cell purification include fluorescence activated cell sorter (FACS) isolation or magnetic cell sorting. These methods yield an isolated transgenic population of PGCs, which are then transferred to a recipient egg.

Donor gonadal PGCs are derived from the same or from a different breed of bird from the recipient egg. In the latter case, identification of donor PGC-produced germline chicks is facilitated by on a difference in distinguishing characteristics of the breed, e.g., color.

### EXAMPLE 2

### Recipient Eggs

Fertilized laid eggs are used as recipient eggs. Recipient eggs are incubated immediately or soon after laying or stored cold (unincubated) after laying but prior to transfer. Incubation begins upon exposure of the eggs to a warm temperature which is conducive to further development of the embryo (e.g., 37-38° C.). A “0 hour” egg is one that is laid but has not yet been incubated. Similarly, a “12 hour” egg is one that has been incubated for 12 hours and so on. Fertilized eggs for recipients are used at 12, 24, 48, and up 96 hours from the start of incubation.

The developmental stage of the recipient egg is determined by chronologic age or hours post-incubation (see, e.g., Hamburger & Hamilton, 1951 J Morphol 88: 49-92) or by visual inspection of the stage of development of embyonic tissues. PGCs are transferred to recipient eggs in a volume of 2-20 microliters. Approximately 50-1000 PGCs are transferred. For example, PGCs are transferred at a cell density of about 50-400 cells/microliter. The site of transfer will depend on the developmental stage of the egg. At 0-12 hours of incubation, the site of transfer is into the blastodermal disc or into the blastocoel cavity. The volume in which donor PGCs are transferred is generally less than 5 microliters of medium. At 24 hours (stage 7-8, Hamburger & Hamilton, 1951 J Morphol 88: 49-92), the site is off center (the margin between the area pellucida and the area opaca) so as not to physically affect the developing chick. At 48 hours (stage 11-13), the site of transfer is at or near the germinal crescent region where PGCs normally congregate prior to entering the vasculature and migrating to the gonads. Alternatively, the PGCs are injected straight into the vasculature at around 50-60 hours from the start of incubation or from stage 13-17 (Yasuda etal., 1992 J. Reprod and Fert 96:521-528, Naito, etal. 1998, J. Reprod and Fert 113: 137-143).

For example, PGCs are transferred to a recipient egg at stage 7-12 of development in which the blastocoelic cavity has been formed following short term incubation of the egg for 6-12 hours. In this case, the PGCs are injected into the blastocoelic cavity. If the recipient egg is between stages 13-16 of development (egg incubation time of 24-28 hours, the PGCs are transferred in the region between the area pellucida and the area opaca. If the recipient egg is at a stage of development corresponding to 36-48 hours of incubation, the PGCs are injected directly into the germinal cresent region, where endogenous PGCs normally congregate prior to entering the chicken vasculature. If the recipient egg is at a stage of development corresponding to 50-72 hours (stage 16-19), the PGCs are transferred directly into the vasculature, e.g., into a blood vessel.

To increase the rate of germline transmission of the donor PGCs, the endogenous PGCs are physically removed from the bloodstream (Naito, etal. 1998, J. Reprod and Fert 113). Alternatively, the egg is irradiated between 0-24 hours of incubation to eliminate the endogenous PGCs (Carsience et.al 1993, Development 117: 669-675). Chemical sterilization using busulphan is also effective to eliminate endogenous PGCs (Aige-Gil & Simkiss 1991, Research in Veterinary W science 50: 139-144; Vick etal, 1993, J. Reprod and Fert 98: 637-641). Dimethyl formamide (20-50 microliters) or a combination of busulfan (100 mg/ml) dissolve in dimethyl formamide and mixed with sesame oil as the carrier, injected into the yolk between 20-24 hours post incubation (75-100 μg busulphan per egg in 50 microlitters of sesame oil) is also effective in killing endogenous PGCs. Alternatively, 50 μg of busulphan in 10-20 microlitters of media are injected in the germinal crescent region (where the PGCs accumulate prior to entering the vasculature). Twenty-four hours post sterilization, the donor PGCs are transferred to the germinal crescent area at 48 hours of incubation or into the vasculature at a later time (between 55-72 hours).

Following transfer, the eggs are incubated at 37-38° C. in a humidified incubator to develop to term and grow to sexual maturity. When the breed of bird is different between donors and recipients, the resulting bird (containing heterologous DNA) is bred to the same breed as that from which the donor PGCs were derived. This approach allows one to distinguish those birds derived from endogenous PGCs and from those derived from donor PGCs (based on distinguishing characteristics such as size, color, etc.).

### EXAMPLE 3

### Improved Avian Transgenics using Gonadal PGCs

There are two scenarios for germ-line transmission of introduced heterologous nucleic acid sequences: (i) target the stage of development prior to embryonic differentiation or (ii) direct genetic manipulations post differentiation by targeting integration events to the germ cell population. Using the latter approach, procedures were designed to produce germ line chimera parent stocks with high rates of germline transmission derived from the transfer of germ cells in their primordial state. The avian approach offers the biopharmaceutical industry a more efficient platform for large-scale manufacturing capabilities compared to other transgenic systems.

Gonadal PGCs are targeted for germline transmission of gene constructs designed for expression in the transgenic chickens during egg formation. Transfection of cultured PGCs was targeted in vitro and subsequently transferred to targeted regions in the developing chick embryo.

The following materials and reagents were used in the described methods: Egg incubator, CO₂ incubator, Stereomicroscope (1-5×), Compound microscope (10×, 20×), Centrifuge, Caliper controlled injector system with pipette holders, Micromanipulator system, 2 #5 watchmaker's forceps, Adjustable pipettes (10, 200, 1000 microliters), Egg Shell cutter (i.e. Dremel Multipro Kit #3956), Petridishes (35, 100 mm), 4-well Nunclon plates, Microcentifuge tubes (1.5 ml), Parafilm (1″ width), Busulphan (Sigma), Sesame oil (Sigma), Dimethylformamide (Sigma), Phosphate Buffered Saline, DMEM hi-glucose (Gibco-BRL), Trypsin/EDTA, Lipofectamine (Gibco-BRL), Ficoll (Sigma), Fetal Bovine Serum, Chicken Serum, Growth Factors (IGF-1, bFGF, mLIF, SCF), Antibiotics (Pennicillin/Streptomycin), Fertilized, SPF eggs (Charles River Laboratories)

Preparation of Donor Primordial Germ Cells

Gonads were isolated as follows. Freshly laid fertilized chicken eggs were incubated for up to 8 days in a humidified egg incubator at 37-38° C. and between 85-88% relative humidity. Gonads were harvested from the developing chick embryo between days 4-8 (stage 29-36). Preferably, gonads are harvested between days 7-8 (stage 31-36) thereby maximizing the number of PGCs recovered with minimal loss of migratory capabilities when transferred back to recipient chick embryos. The gonads were recovered by removing the mesonephros region from the abdominal cavity of the embryos and dissecting out the gonads from the mesonephros using fine tip forceps under low power magnification. At 7-7.5 days of incubation, developmental differences between the differentiating female and the male gonads can be identified allowing for sex selection. The developing pair of female gonads shows atrophy of the right gonad and an enlarging left gonad resulting from the differential colonization pattern of the germ cells. The differentiating male gonads are similar in size and are distinctive from the female gonads. Although gonads can be isolated at later stages of development with more distinctive differences between the male and female gonads, the ability of isolated PGCs to migrate is reduced when transferred to recipient chick embryos.

Donor PGCs can be derived from the same breed or from a different breed of bird than the recipient egg. In the latter case, identification of donor PGC-produced germline chicks is facilitated by differences in distinguishing characteristics of the breed (e.g. feather color, size and skin pigmentation). It is preferable that the source of the PGCs phenotypically expresses a different feather color than the recipient chick embryo. This facilitates easier selection of chicks derived from the transferred PGCs.

Isolation and Culture of the Germ Cells

The isolated gonads were grouped by sex and dispersed by standard trypsinization procedures. The PGCs were distinguished by their large size (12-20 microns) compared to the gonadal stromal cells. Under bright field microscopy, the PGCs contain numerous lipid droplets throughout the cytoplasm with the large nucleus occupying an eccentric location. Chicken PGCs have high glycogen content and thus are identifiable by periodic acid Schiff staining. The PGCs can either be co-cultured with the gonadal stromal cells or separated prior to culture on plates at 37° C. in a humidified incubator with 5% CO₂ for a period of up to 4 days. In some cases, long term culture may result in spontaneous differentiation, loss of migratory capability and reduction of germ cell potential. The PGCs were cultured in DMEM with high glucose content and supplemented with 10% FBS, 5% chicken serum and growth factors (basic Fibroblast Growth Factor, Insulin Growth Factor-1 and Stem Cell Factor at 10 ηg/ml and murine Leukemia Inhibitory Factor at 10 units/ml) to maintain their germ cell state. The PGCs were separated from the gonadal stromal cells using a ficoll density gradient. For the gradient, a 1.5 ml centrifuge tube was sequentially layered with 0.5 ml each of 16% and 7% ficoll in PGC media and overlaid with a 0.2 ml gonadal cell suspension. The gradient was centrifuged at 800×g for 30 min. The PGC-rich fraction located between the 16% and 7% gradient was aspirated, washed with PGC media and pelleted at 500×g for 5 min. Further purification is accomplished by short-term culture (15-30 minutes) of the isolated cells allowing differential attachment of gonadal stromal cells to tissue culture plates while the PGCs remain in suspension.

Transfection and Selection of Transgenic PGCs

The PGCs were transfected using lipids (i.e. Lipofectamine, according to vendors instructions) for 3-4 hours or by electroporation from 200-250 volts and between 750-950 microfarads with 20-50 μg/ml of DNA.

Two test transgenes were transfected. Using these methods, beta galactosidase under the control of the Cytomegalovirus promoter was used as a test transgene to track gonadal colonization patterns of the donor PGCs. Additionally, a human lactoferin promoter sequence was used. Recipient chick embryos were recovered at day 10 of incubation showing expression of the test transgene beta galactosidase in the mesonephros-gonadal region. Donor PGCs were transfected in vivo using direct blastodermal disc injection prior to egg incubation, or in vitro using lipids. PGCs were transferred to the germinal crescent region of a 48 h incubated egg that was partially sterilized with 75 micrograms of busulphan 24 h prior to PGC transfer.

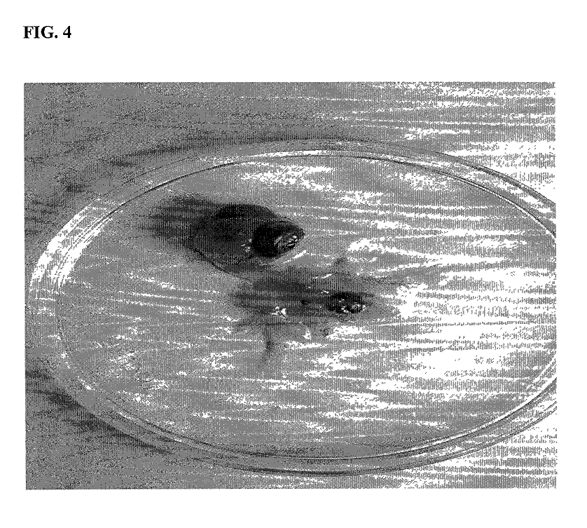

Using these methods, a recipient chick embryo was recovered at day 10 of incubation showing expression of the test transgene beta galactosidase in the mesonephros-gonadal region (FIG. 4). Donor PGCs were transfected in vitro using lipids and transferred to the germinal crescent region of a 48 h incubated egg that was partially sterilized with 75 micrograms of busulphan 24 h prior to PGC transfer. Beta galactosidase expression was under the control of the Cytomegalovirus promoter.

A range 10 to 20% initial transfection rate was obtained using the above methods. A combination of multiple rounds of transfection and selection is optionally incorporated to increase transfection rates. The transfected PGCs were separated and purified by antibiotic selection if the transgene construct carries a marker encoding antibiotic resistance. Other methods of cell purification can be used including fluorescence activated cell sorter (FACS) if a fluorescent marker is used for selection.

Preparation of Recipients

Fertilized eggs up to 7 days post oviposition were used as recipient eggs. Recipient eggs prior to, and up to 72 hours of incubation (up to stage 19), were utilized as PGC recipients. This time period includes the stages of chick development where the PGCs are actively migrating, and up to the time of localization in the primordial gonads. PGCs at a concentration of 100-200 per microliter were injected using a glass micropipette (25-40 microns) attached to a Hamilton syringe under the control of a micrometer plunger. A volume of 5 microliters of PGCs suspended in culture medium was injected. PGCs may be transferred to different targeted regions depending on the stage of development of the chick embryo: (a) into the subgerminal cavity of the blastodermal disc of fertile non-incubated eggs, (b) into the blastocoel cavity that separates the epiblast and the hypoblast of the developing embryo incubated for 6-12 hours (stage 3-4), (c) into the area pellucida adjacent to the developing embryo at 24-28 hours of incubation (stage 7-8), (d) into the germinal crescent region apical to the head process of the developing chick embryo incubated between 40-48 hours (stage 11-13), (e) 400-600 PGCs in 2-3 microliters of media are injected into the vasculature at 55-72 hours of incubation (stage 14-19) using a glass micropipette (40 microns outside diameter) attached to a micromanipulator. Preferably, the PGCs are injected into the dorsal aorta although larger marginal veins and arteries can be used as sites of injection. At this stage, PGCs circulate normally within the vascular system prior to migrating to the gonadal anlage.

To increase the rate of germline transmission of the donor PGCs, the circulating endogenous PGCs are physically removed from the bloodstream by aspirating the blood at the time when PGCs are in the vasculature, thus allowing partial sterilization or the egg irradiated between 0-24 hours of incubation to eliminate the endogenous PGCs. Chemical sterilization using busulfan may also be used. Busulfan is dissolved in Dimethylformamide and 75 μg in 50 microliters of sesame oil is injected into the yolk of recipient eggs 20-24 hours after the start of incubation to reduce endogenous PGC involvement. Twenty-four hours post sterilization, the donor PGCs were transferred to the germinal crescent region, 48 hours from the start of incubation or into the vasculature 55-72 hours from the start of incubation. Following transfer, the eggs were incubated at 37-38° C. in a humidified incubator until hatching.

Generation of Transgenic Germline Chimeric Embryos

Recipient chick embryos were recovered at day 10 of incubation showing expression of the test transgene beta galactosidase in the mesonephros-gonadal region. Polymerase chain reaction results (PCR) from gonads isolated from day 14 recipient embryos showed strong positive signals for the integration of the human lactoferin promoter sequence when donor PGCs were isolated and then transfected in vitro. A weaker positive signal was obtained from gonads of recipients when donor PGCs were transfected in vivo.

Generation of Chicks from Donor PGCs

When the breed of the bird is different between the donor PGCs and recipient embryo, the resulting germline chimeric bird is bred to the same breed as that from which the donor PGCs is derived. This approach allows one to distinguish those birds derived from endogenous PGCs from those derived from donor PGCs. The distinguishing phenotypic characteristics that can be attributed to a specific breed such as feather color allows easy identification of donor PGC derived chicks. For example, feather color identification of chicks indicated whether they were produced from donor derived PGCs (black feathers) or endogenous PGCs (white feathers). The addition of a sex-linked trait further allows the selection of the males (e.g., white patch on the head) from the females.

FIG. 4 shows expression of a test transgene beta galactosidase. Table 2 shows the results of PGC-mediated transfer of a second transgene, human lactoferrin. Transfection of PGCs was carried out in vitro as described above or in vivo at day 0 of incubation (U.S. Ser. No. 09/587,128; hereby incorporated by reference). In vivo transfection was carried out by introducing DNA directly into the germinal disc of a first egg at day) of incubation. The egg is then incubated for 7.5 days and the PGCs removed, cultured, and transferred to a second (recipient) egg. PGCs may be transfected again with transgene DNA in culture. Tranfected PGCs were then transferred to the germinal crest region (GCR) of a recipient egg. The recipient eggs were incubated, and day 14 chick embryonic tissues were tested for presence of the transgene by polymerase chain reaction (PCR).

TABLE 2
Detection of human lactoferrin DNA in d14 chicks

| Exper- | Sample/ | | | | |
|---|---|---|---|---|---|
| iment | Tissue | PGC Recovery | Transfection | Transfer | Result |
| 1 | 1. Gonads | 6.5-7.5 days | In vitro | GCR | Neg. |
| | 2. Bodies | 6.5-7.5 days | In vitro | GCR | Neg. |
| | 3. Control | 6.5-7.5 days | In vitro | GCR | Pos. |
| | 4. Gonads | 6.5-7.5 days | Day 0 In vivo | GCR | Neg. |
| | 5. Bodies | 6.5-7.5 days | Day 0 In vivo | GCR | Neg. |
| | 6. Control | 6.5-7.5 days | Day 0 In vivo | GCR | Pos. |
| 2 | 1. Gonad | 6.5-7.5 days | In vitro | GCR | Pos. |
| | 2. Bodies | 6.5-7.5 days | In vitro | GCR | Pos. |
| | 3. Control | 6.5-7.5 days | In vitro | GCR | Pos. |
| | 4. Gonads | 6.5-7.5 days | Day 0 In vivo | GCR | Weak |
| | | | | | Pos. |
| | 5. Bodies | 6.5-7.5 days | Day 0 In vivo | GCR | Weak |
| | | | | | Pos. |
| | 6. Control | 6.5-7.5 days | Day 0 In vivo | GCR | Pos. |
| 3 | 1. Gonads | 7.5-8.5 days | Day 0 In vivo | GCR | Neg. |
| | 2. Gonads | 7.5-8.5 days | In vitro | GCR | Neg. |

Advantages of an Avian System

An avian system allows expression of the gene product in the albumen or yolk fraction of the eggs or other tissues of the bird. The avian species, specifically the chicken, offers an inherent advantage over most if not all of the domestic livestock species currently available as target production systems. Its reproductive potential and short generation time is a potential advantage compared to the other species presently utilized for transgenic production.

The methods described herein offer an alternative assisted reproduction technology targeted towards the chicken where genetic modifications are directly targeted towards the germ cell population. Using this approach, methods for foreign gene integration in the germ-line of birds is enhanced through technologies that improve the frequency of stable integration events. These include proper nucleic acid sequence construction with efficient vectors and promoters for targeted expression and improving gene delivery efficiency by multiple rounds of in vitro transfection of germ cells combined with methods to identify and isolate stable integrants prior to transfer to recipient chicken embryos.

The methods provide a system to produce and differentiate between chicks derived from donor PGCs or endogenous PGCs following partial sterilization. The technology produced birds derived from donor PGCs with a high rate of germline transmission ranging from 25-78% where almost half of the chicks produced (49%) on average were donor derived. Incorporating feather color as a visual marker, additionally offers a mechanism to identify and differentiate donors from endogenous chicks, simplifying the production system. Furthermore, the procedure eliminates the need to run DNA analysis on every chick produced following breeding. This saves time and resources thus reducing cost of producing germline transgenic chickens. Additionally, when specific feather coloration is attached to a sex-linked trait, the chick is easily sexually identified, separated and grown in order to maximize the production efficiency based on the potential of the two sexes. For example, when a desired trait or gene product is targeted specifically for expression towards egg production, a sex-linked feather trait allows early selection of production birds and elimination of unwanted birds when chicks are only a few days old. Furthermore, expression profiles are established from individual female birds within a month of their hatching providing realistic production projections. All of the above factors facilitate increased management efficiency equating to a reduction in overall production costs to an inherently low production cost animal.

### EXAMPLE 4

### Avian PGCs for Transgenesis and Conservation

PGCs are the progenitors for sperm and oogonia. In the avian species, PGCs are extra-embryonic in origin and migration is a combination of active migration to the germinal crescent region, followed by a passive stage where they temporarily circulate in the vascular system prior to an active migration to the gonadal anlagen where PGC proliferation and gonadal sex differentiation occurs.

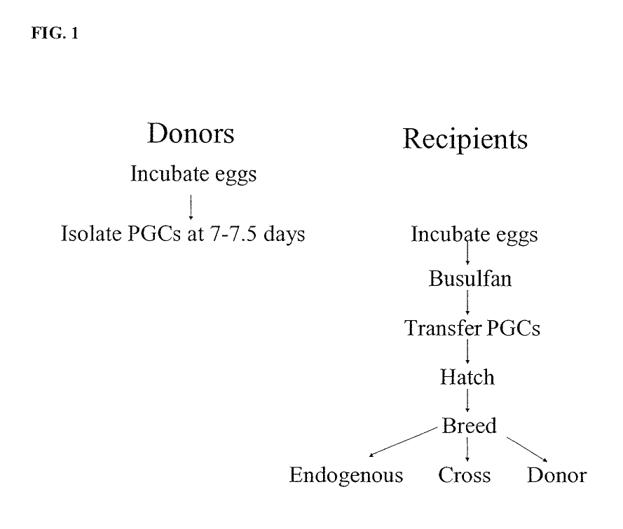

In chickens, sex differentiation becomes morphologically evident between 7-9 d of embryonic development. The point of sex differentiation of other avian species is determined by visual inspection of the gonads. This stage of gonadal development in chickens was targeted in order to: a) maximize the number of PGCs recovered, b) determine whether PGCs still retain migratory capacity at this stage where morphological differences in gonadal sex development is evident, c) determine the effects of breed dominance in germline transmission and d) study the effects of opposite sex transfer on the germline transmission of PGC donor-derived chicks. A flow chart of the procedure is shown in FIG. 1. Gonads from 6.5-8.5 d embryos were isolated and morphologically separated by sex. PGCs were isolated by standard trypsinization procedures and mechanical disruption. PGCs were co-cultured with their gonadal stromal cells at 37° C. in 5% CO₂ for 2 d in DMEM with high glucose in the presence of 10% FBS, 5% chicken serum supplemented with antibiotics and growth factors (bFGF, IGF-1, SCF at 10 ηg/ml and mLIF at 10 U/ml). The PGCs were isolated in a 1.5 ml centrifuge tube using a ficoll gradient sequentially layered with 0.5 ml each of 16% and 7% ficoll overlaid with 0.2 ml of cell suspension and spun for 30 min at 800×g. To generate germline chimeras, approximately 2000 PGCs were transferred at 44-48 h into the area pellucida and the germinal crescent region of recipient eggs treated with 75 μg of busulfan at 24 h of incubation. Chicks derived from donor PGCs were distinguished by feather color differences between the breeds used as PGCs donors and the embryo recipients.

Germline transmission of donor-derived chicks ranged from 31-78% between experimental groups with an average transmission rate of 49% (97/198) when parent stocks were bred together. Of this, 92% of the chicks were crosses and 8% were pure donor derived chicks. When PGCs were derived from White Leghorn embryos and transferred to colored breeds, germline transmission rates were 47% compared to 17% when donor PGCs from either Rhode Island Reds or Barred Plymouth Rocks were transferred to White Leghorn embryos. There was also a higher rate of transmission when the recipients received the same sex PGCs (40%) compared to the opposite sex (7%).

Integrating the ability to introduce heterologous nucleic acid sequences into PGCs combined with efficient selection of stable integrants provide a platform technology for production of biopharmaceuticals. Furthermore, this reproductive technology is useful to improve production breeds, confer disease resistance, and increase production efficiency in avian agriculture. Additionally, PGC transfer is useful for avian conservation programs. For conservation purposes, the chicken, a non-seasonal highly productive bird, is used as a “universal recipient”, providing alternative systems to enhance captive breeding programs for conservation year round.

Unlike mammalian PGCs, avian PGCs are extra-embryonic in origin. At the time of oviposition in chickens, there are roughly about 50 PGCs interspersed within the 40-60 thousand cells comprising the blastodermal disc of a fertilized oviposited egg. Their migratory pattern follows a circuitous path involving a combination of active and passive migratory phases. During the first 24 hours of egg incubation, approximately 500 cells in the Area Pellucida of the blastodermal disc converges to establish the posterior end of the developing embryo. From the convergence, the primitive streak develops towards the anterior end in direct apposition to the area of convergence. Also at this time, the PGCs actively migrate away from the convergence and towards the anterior end, independent of the developing embryo and congregating in the germinal crescent region above the head fold prior to entering the vasculature at about 2-2.5 days of incubation. Subsequently, the PGCs passively migrate within the circulatory system until they reach the genital ridge where they leave the vasculature and actively migrate and colonize the developing gonads.

Several methods have been described directly targeting the PGCs for transgenic manipulations in vivo on the basis of their spatio-temporal localization during their migratory phase or post colonization of the gonadal anlagen. Some of the approaches involve directly injecting the lipid encapsulated heterologous nucleic acid sequences into the germinal crescent region or into the vasculature of the embryo during the passive migratory phase. Alternatively, methods have also been developed to recover PGCs from the germinal crescent region, the vascular system, and from the gonads prior to sex differentiation. The methods described herein involve directly targeting gonadal PGCs at a later stage of embryonic development compared to previously described protocols and after sex differentiation. The results described herein were surprising, because similar to the findings in mammals, earlier reports indicated that PGCs lose their migratory capacity immediately following gonadal localization. The data indicated that purified sex-selected PGCs migrate following transfer to the germinal crescent region of a recipient egg. It was unexpected that PGCs at the later stage of embryonic development still retain migratory capacity especially after genetic differentiation of sex had been established.

Manipulations were targeted at a specific stage of embryonic development in chickens in which early morphological differences between the male and female gonads become evident. The study was designed (a) to determine whether PGCs still retain migratory capacity at this stage of gonadal sex development; (b) to maximize the number of PGCs that can be recovered without loss of migratory capacity; (c) to determine whether different breeds exhibit breed dominance relative to germline transmission when donor PGCs are different from the breed of the recipient embryo; and (d) to determine whether transferring PGCs to recipients of similar or opposite sex have any influence in the rates of germline transmission.

Preparation of Donor Primordial Germ Cells

Fertilized chicken eggs from Rhode Island Red, Barred Plymouth Rock and White Leghorn were incubated up to 8 days in a humidified egg incubator at 37-38° C. and between 85-88% relative humidity. Gonads were isolated by harvesting the mesonephros from the genital ridge and dissecting out the gonads from the mesonephros using fine tip forceps under low power magnification. Between 7-7.5 days of incubation, the developing gonads in females exhibit a slightly larger left gonad compared to the right. The differentiating male gonads on the other hand are similar in size providing the morphological distinction between the two sexes The gonads were grouped based on breed (white or colored breeds) and by sex, and dispersed by standard trypsinization procedures. The PGCs were co-cultured with the gonadal stromal cells in tissue culture plates at 37° C. in a humidified incubator with 5% CO₂ for up to 4 days. The PGCs were cultured in DMEM as described in Example 3 above.

The PGCs, which are larger (14-20 microns) compared to the other gonadal stromal cells, were separated using a ficoll density gradient as described in Example 3 above.

Periodic Acid-Schiff Staining

Gonadal cells were cultured in 4-well plates and fixed in 3.7% formaldehyde for 15 minutes and washed in PBS twice. Chicken PGCs exhibit high levels of glycogen and are readily identified using Periodic Acid-Schiff staining. The Periodic Acid-Schiff staining system supplied as a kit (Sigma Diagnostics) was used to differentiate PGCs from the stromal cells that comprise the rest of the gonads.

Preparation of Recipients

On the day of PGC harvest, fertilized eggs from White Leghorn or Rhode Island Red were incubated to serve as recipients. To reduce endogenous PGC involvement, the chemo-sterilant busulfan is optionally used to partially sterilize the recipients. Busulfan was dissolved in Dimethylformamide and at 24 hours after the start of incubation, 75 μg of Busulfan in 50 μl of sesame oil was injected into the yolk of recipient eggs. Between 24-30 hours post sterilization, the donor PGCs were injected into the germinal crescent region apical to the head process of the developing recipient embryo. A volume of 5-10 μl of PGCs (100-200 PGCs per μl) suspension was injected per egg using a 50-micron (OD) glass micropipette attached to a micrometer-controlled Hamilton syringe.

Following transfer, the eggs were sealed with parafilm and incubated at 37-38° C. in a humidified incubator for an additional 18 days and transferred to a hatching incubator to hatch.

Generation of Chicks from Donor PGCs

A breeding pair randomly selected from each of the 6 experimental replicates was used as chimeric parent stocks. White leghorn recipients with Rhode Island Red and Barred Rock donor PGCs were bred with each other, and Rhode Island Red recipients with White Leghorn Donor PGCs were bred together. The birds were pen mated and eggs derived from each breeding replicate were hatched on a weekly basis. First generation chicks were identified as donor-derived or endogenous PGC-derived based on the characteristic feather coloration attributed to the breed of the birds or the crosses derived from them.

Results and Statistical Analysis

Chi-Square method was used to analyzed the data at a significance level of <0.05.

The average hatching rates of recipient eggs was 37% from a range of 9-67%.

First generation chicks identified by feather markings as being derived from donor PGCs ranged from 17-71% among the breeding groups with an average transmission rate of 49% (97/198. Data from additional hatchings are shown in Table 3.

TABLE 3
Chicks produced from the pairing of PGC recipients

| Experiment number | 1 | 2 | 3 | 4 | 5 | 6 |
|---|---|---|---|---|---|---|
| Total number of | 147 | 103 | 140 | 29 | 17 | 12 |
| chicks | | | | | | |
| PGC-derived chicks | 84 | 63 | 24 | 8 | 13 | 6 |
| Total (%) | (57) | (61) | (17) | (28) | (76) | (50) |
| PGC-derived chicks | 80 | 55 | 24 | 7 | 11 | 6 |
| Cross (%) | (95) | (87) | (100) | (87) | (85) | (100) |
| PGC-derived chicks | 4 | 8 | 0 | 1 | 2 | 0 |
| Pure (%) | (5) | (13) | (—) | (13) | (15) | (—) |

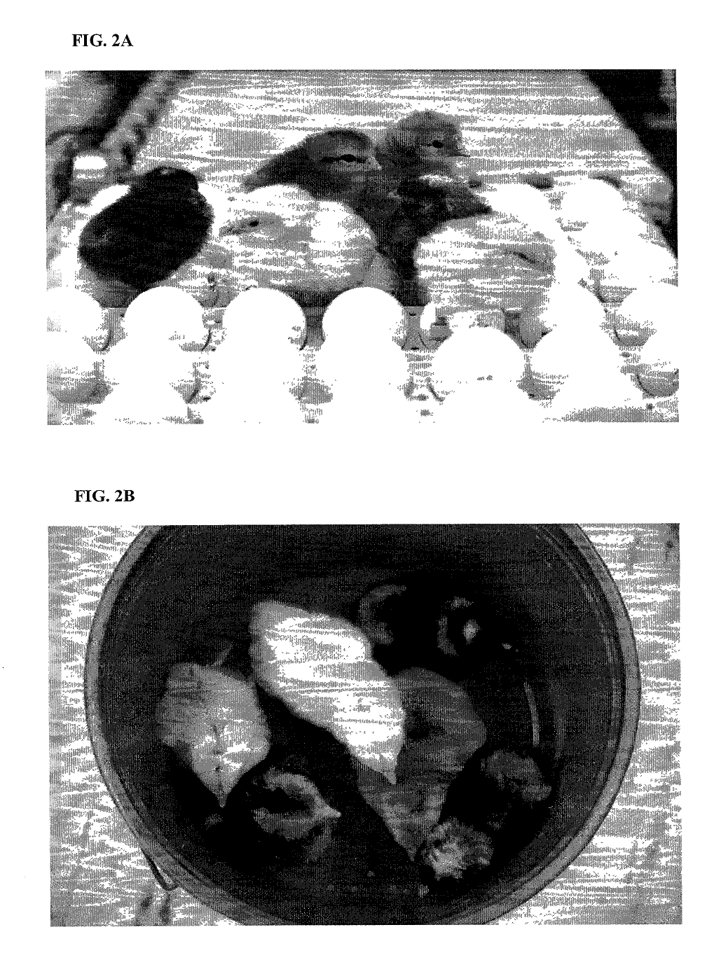

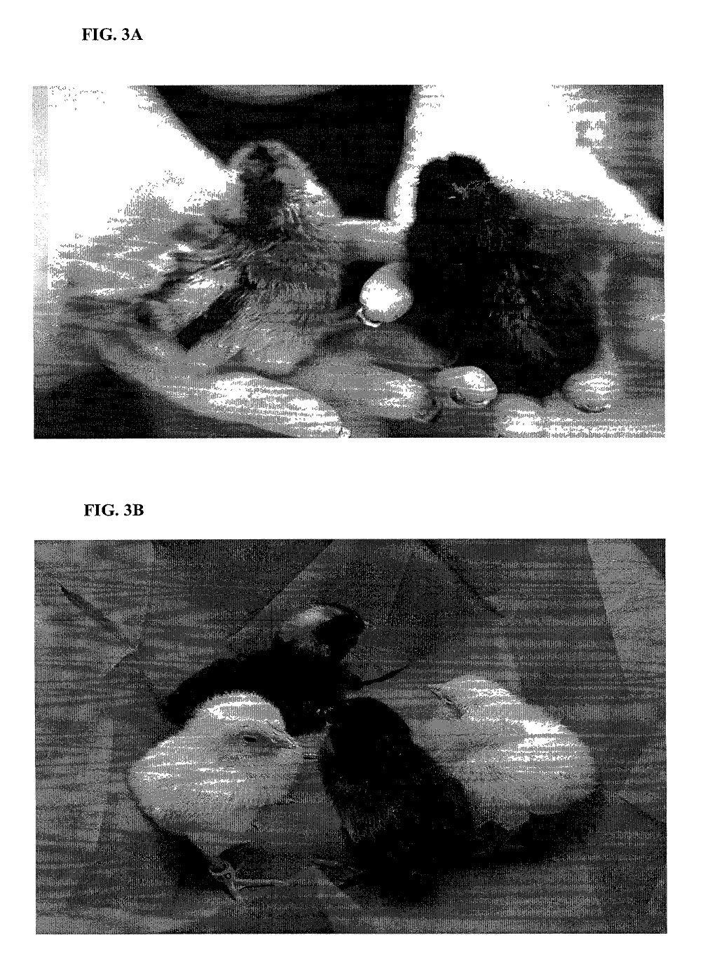

The mating system using both male and female recipients as breeding pair increased the rates of transmission and also resulted in pure donor PGC-derived chicks. Of the chicks identified as being donor-derived, 85% were from crosses between donors and endogenous germ cells and 15% were pure donor-derived chicks (FIGS. 2A, 2B). The breeding scheme using White leghorn recipient breeding pairs receiving Rhode Island Red and Barred Rock PGCs produced 3 crossbreds and 3 purebred strains. Furthermore, a sex-linked feather trait was observed when pure donor-derived chicks were obtained from a breeding combination of PGCs derived from Rhode Island Red and Barred Rock (FIGS. 3A, 3B).

When PGCs were derived from White leghorn embryos and transferred to colored recipient, germ line transmission rates were 47% compared to 17% when colored PGCs were transferred to White Leghorn recipients.

When both recipients and PGCs were of similar sex, germline transmission rates were 40% compared to 7% when recipients received PGCs of the opposite sex.

Targeting the manipulations at a specific stage of chicken embryonic development where morphological differences between the male and female gonads were evident, indicated that PGCs regardless of sex still retain migratory capacity when transferred into the germinal crescent region of recipient chick embryos. This result was surprising in view of earlier reports. An additional advantage of this procedure is that more PGCs are recovered from the gonads than can be obtained at earlier stages of development

The results obtained in this study may suggest that PGCs from White Leghorn have higher (<0.05) transmissibility compared with the germline involvement of both Rhode Island and Barred Rock. Although this may be attributed to breed dominance, other factors such as developmental differences (Rhode Island and Barred Rock are early maturing compared to the White Leghorns) rather than breed dominance may be involved. Additionally, transmission rates appeared to be higher (<0.05) when the sex of the PGCs was similar to the recipients.

The data indicated that the methods described herein represent an alternative assisted reproduction technology for avian agriculture that provides for efficient germline manipulation to improve production breeds, confer disease resistance and production efficiency. Transgenic animals are produced by introducing heterologous nucleic acid sequences under the control of appropriate regulatory promoters directly into the germ cell population and selecting stable integrants prior to transferring transgenic PGCs into recipient eggs. A significant advantage of this system is that it can be used to enhance flock management and reduce overall production cost by incorporating feather color to identify chicks derived from manipulated germ cells, and select them at day old using sex-linked feather color trait.

As was discussed above, the PGC method also provides an alternative assisted reproduction technology to enhance avian conservation programs, because the chicken, a non-seasonal highly productive bird is used as a “universal recipient” providing alternative systems to enhance captive breeding programs for avian conservation year round.

### EXAMPLE 5

### Avian PGCs as Stem Cells for Somatic Tissue Generation

Stem cells are the primordial units of embryonic generation and adult regeneration. Their intrinsic capacity to respond to extrinsic signals regulates their lineage fate. In mammals, primordial germ cells (PGCs) have the ability to remain undifferentiated and continuously proliferate in culture as well as exhibit multipotent capacity to participate in the formation of the three embryonic germ layers.

The data described herein indicates similarities between mammalian and avian PGCs and shows that chicken PGCs in the early stages of gonadal sex differentiation still retain stem cell potential and can differentiate spontaneously in vitro and contribute to various tissues of the developing embryos in vivo after embryonic lineage differentiation has been established.

PGCs exhibited a tendency to differentiate towards the neural and mesenchymal lineages. Using a fluorescent transgene marker (DsRed) to provide an unambiguous tag, observable localization patterns and extent of multipotency and plasticity in vivo were established in the developing fetus. The data indicate that isolated PGCs from sexually differentiating 6.5-8.5 d chicken gonads had not committed to an irreversible somatic lineage and were still capable of contributing to all germ layers in vivo. Furthermore, the vasculature in chickens is not limiting, but facilitates the wide spread localization of PGCs and their subsequent multilineage involvement in various tissues of the developing fetus. The gonadal PGCs described herein retained the intrinsic capacity to respond to extrinsic signal regulators for multilineage differentiation.

Experiments were designed (a) to determine whether chicken PGCs in the early stages of gonadal sex differentiation have already been committed to its somatic lineage or whether they still retain stem cell potential and differentiate into other somatic lineages in vitro and various tissues of the developing embryos in vivo; (b) to determine whether the sex of the PGCs will affect their lineage involvement; and (c) to determine whether the vascular system in chicken embryos provides an acceptable route for tissue colonization.

Isolation of the Gonads

Freshly laid fertilized Barred Rock eggs were incubated up to 8.5 days in a humidified egg incubator at 37-38 ° C. and between 85-88% relative humidity. Gonads were harvested from the developing chick embryo between 7-8 days of incubation (stage 31-34). The gonads were recovered by removing the mesonephros from the genital ridge and dissecting out the gonads from the mesonephros using fine tip forceps under low power magnification. The gonads were grouped according to sex. Females have a larger left gonad compared to the right, while male gonads exhibit a similar size.

Culture of Germ Cells

The PGCs were isolated following standard trypsinization procedures and cultured with or without the gonadal stromal cells in tissue culture plates at 37° C. in a humidified incubator with 5% CO₂. The PGCs were cultured and isolated using a ficoll density gradient as described above. Periodic Acid-Schiff staining was carried out as described above.

Transfection of PGCs

To provide an unambiguous tag for assessing in vivo localization pattern and lineage involvement, the PGCs were transfected with Ds Red (Clontech Laboratories) to express cytoplasmic red fluorescence. Lipofectamine (GIBCO-BRL) at 15 μl was diluted to a final volume of 10 μl in OPTI-MEM 1 (GIBCO-BRL) and 1 μg of linearized DNA (Ds Red) was also diluted to the same final volume in OPTI-MEM 1 (GIBCO-BRL). The preparations were combined and incubated for 30 minutes at room temperature and then added drop-wise into the wells. Culture media was added after 4 hours to inhibit toxicity. Consecutive rounds of transfections were used to improve the rate of transfection.

In Vitro Differentiation

To establish pluripotential capacity in vitro, the PGCs were removed from gonadal-stromal co-culture and induced to spontaneously differentiate in the absence of growth factor supplements.

Production of Somatic Chimeras

Mltipotency and plasticity in vivo was determined using 2.5-3.5 day old White Leghorn embryos as recipients. Prior to transfer, the eggs were windowed at the blunt end to visualize the developing chick embryo. About 400-600 PGCs in 2-3 μl of media were injected into the vasculature between 60-84 hours of incubation using a glass micropipette (40 microns OD) attached to a micromanipulator. The PGC suspension was injected into the vasculature, e.g., at the dorsal aorta or the marginal veins and arteries of the developing embryos. The egg window was sealed and the eggs were incubated at 37° C. in a humidified incubator. PGC localization and multilineage involvement was determined between 7-9 days of fetal development using the fluorescent Ds Red marker and at day 1 after hatching to also determine feather color influence.

Culture Characteristics of Chicken Germ Cells

Colonies proliferated over 3 months in repeated subcultures in the presence of mitotically active gonadal stromal cells. Multipotent germ cells were uniformly round and did not attach tightly to the stromal layers or to other PGCs. Colonies were multi-layered and well delineated from the stromal layers. Loss of monolayer support led to some level of spontaneous differentiation even in the presence of both inhibitory and growth factor supplements. Long-term culture also showed a decrease in Periodic Acid-Schiff staining intensity.

Multipotency in vitro

In the absence of gonadal stromal support and growth and inhibitory factor influence, PGCs differentiated spontaneously into several cell lineages. The majority of the PGC cultures differentiated into fibroblast-like and mesenchymal type cells. Extended cultures of these cells subsequently led to a variety of connective-tissue cell types morphologically similar to bone, cartilage, muscle and immature fat cells. When PGCs proliferated into small compacted clusters of cells with minimal or loose monolayer attachment, they often differentiated into neural cells forming extensive network between them. PGCs also differentiated occasionally into an epithelial monolayer at confluence.

Multipotency and Plasticity in vivo

Using the vascular system as the route of transfer facilitated extensive localization and lineage involvement of PGCs in the developing chicken fetus. They were consistently observed in the heart, lungs and blood; and were estimated based on Ds Red expression to have contributed up to 20, 5 and 40% of the heart, lungs, and blood respectively. They were also observed to localize in the midbrain, eyes, bone marrow, muscle, gonads, mesonephros and skin. Lineage involvement was evident by fluorescence in the muscle, skin, bone marrow, gonads, mesonephros and the midbrain at different levels. Feather color patterns of hatched chicks also showed some contribution from the germ cells. Sub-cultured germ cells maintained in the absence of gonadal-stromal monolayer support showed a reduction in multilineage involvement in vivo, specifically loosing their capacity to incorporate into the gonadal involvement.

The data indicate that isolated PGCs from sexually differentiating 7-8.5 day chicken gonads have not committed to an irreversible somatic lineage towards spermatogenesis and oogenesis. These cells still retain the multipotent capacity to differentiate in vitro and exhibit plasticity to contribute into all germ layers in vivo.

There was a high preponderance of both primary and sub-cultured germ cells to differentiate into mesenchymal and neural lineage and subsequently into their terminal somatic state in vitro. No clear evidence that proliferation and lineage involvement was affected when the sex of the donor PGCs was different from the recipient embryos.

The vascular system in chickens is not limiting, but facilitates the wide spread localization of PGCs, allowing them to contribute to tissue generation and organogenesis in the developing fetus. These cells can be harnessed and reprogrammed by extrinsic regulators controlling the environmental milieu that determines their lineage differentiation. Although humans do not share a similar basic body plan to avian or to other animal models used for stem cell research, homologous parts that likely arise from the same biochemical mechanisms are shared. Thus, the chicken PGC model is a useful alternative to other animal models for the development of applications of stem cells in tissue engineering, embryonic generation, adult regeneration and gene therapy.

Other embodiments are within the following claims.

What is claimed:

1. An isolated avian gonadal cell comprising a heterologous nucleic acid.

2. The gonadal cell of claim 1, wherein said cell is an embryonic cell.

3. The gonadal cell of claim 1, wherein said cell is a gonadal primordial germ cell.

4. The gonadal cell of claim 1, wherein said cell is an ovarian cell.

5. The gonadal cell of claim 1, wherein said cell is a testes cell.

6. A method of introducing a nucleic acid molecule into the genome of an avian species, comprising contacting a population of isolated gonadal cells obtained from a chick embryo with said nucleic acid molecule to yield transfected gonadal cells, and transferring said transfected gonadal cells to a fertilized recipient avian egg.

7. The method of claim 6, wherein said population comprises at least 0.5% primordial germ cells.

8. The method of claim 6, wherein said population comprises at least 1% primordial germ cells.

9. The method of claim 6, wherein said population comprises at least 50% primordial germ cells.

10. The method of claim 6, wherein said population comprises at least 90% primordial germ cells.

11. The method of claim 6, wherein said chick embryo is at an embryonic stage of greater than 27.

12. The method of claim 6, wherein said chick embryo is at an embryonic stage of 29-36 of gestation.

13. The method of claim 6, wherein said transfected gonadal cells and said fertilized avian egg are derived from the same species.

14. The method of claim 6, wherein said transfected gonadal cells and said fertilized avian egg are derived from different species.

15. The method of claim 6, wherein said fertilized avian egg is between stage 7-8.

16. The method of claim 6, wherein said fertilized avian egg is between stage 13-19.

17. The method of claim 6, wherein the breed of said chick embryo is White Leghorn.

18. The method of claim 6, wherein the breed of said chick embryo and the breed of said fertilized recipient egg are different.

19. The method of claim 6, wherein said fertilized avian egg is partially sterilized prior to transferring said transfected gonadal cells.

20. The method of claim 6, wherein said fertilized avian egg is contacted with busulfan prior to transferring said transfected gonadal cells.

21. The method of claim 6, wherein said transfected gonadal cells are transferred directly into the germinal crest of said fertilized recipient avian egg.

22. The method of claim 6, wherein the sex of said gonadal cells and the sex of an embryo in said fertilized recipient avian egg is the same.

23. An isolated avian gonadal cell, comprising a genetic disruption of an endogenous gene, wherein said disruption inhibits production of a functional gene product.

24. An avian egg comprising a xenogeneic primordial germ cell.

25. An avian egg comprising the cell of claim 1.
